(19) United States
(12) Patent Application Publication
(10) Pub. No.: US 2005/0008697 A1
(43) Pub. Date: Jan. 13, 2005

(54) Oral solid solution formulation of a poorly water-soluble active substance

(75) Inventor: Henricus R.M. Gorissen, Weesp (NL)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals B.V., Weesp (NL)

(21) Appl. No.: 10/915,560

(22) Filed: Aug. 11, 2004

Related U.S. Application Data
(63) Continuation of application No. PCT/EP03/50014, filed on Feb. 11, 2003.

(30) Foreign Application Priority Data
Feb. 14, 2002 (EP) 02075623.5

(51) Int. Cl. A61K 31/555; A61K 9/20

(52) U.S. Cl. 424/464; 514/184

(57) ABSTRACT

An immediate release pharmaceutical formulation of a poorly water-soluble biologically active substance with enhanced bio-availability, the formulation being a homogeneous and thermostable solid solution, and the solid solution including as percentages of the total weight of the formulation: a) 10 to 50% of the active substance; b) 20 to 70% of a non-ionic hydrophilic surfactant which is liquid between 15 and 30° C.; c) 5 to 70% of a pharmaceutically acceptable organic polymer or polymer mixture which is liquid above 60° C. and solid below 30° C., and d) optionally, 1 to 10% of a disintegrating agent; as well as active substances formulated into such form, and methods for producing such formulations.

### CROSS REFERENCE TO RELATED APPLICATIONS

This application is a continuation of international patent application no. PCT/EP03/50014, filed Feb. 11, 2003 designating the United States of America and published in English on Aug. 21, 2003 as WO 03/068266 A1, the entire disclosure of which is incorporated herein by reference. Priority is claimed based on European patent application no. EP 02075623.5, filed Feb. 14, 2002.

### BACKGROUND OF THE INVENTION

The present invention relates to an oral solid solution formulation for a poorly water-soluble active substance. More in particular the invention relates to a solid solution formulation of a poorly soluble active substance for which the bio-availability is strongly enhanced.

Solid solution formulations, which normally are in the form of gelatine capsules, are known in the art. EP 0001822 describes pharmaceutical formulations in the form of hard gelatine capsules filled with a liquid excipient which contains the active substance and which solidifies into a solid composition or into a thixotropic gel. U.S. Pat. No. 4,795,643 discloses a solid solution formulation with a delayed release of the active substance. The delayed release is caused by the use of special polymers such as acrylate polymers or etherified celluloses.

Various active substances have a very poor solubility in water. When these active substances are administered to the body, they often have a poor bio-availability due to their poor solubility in the digestive fluid. In order to solve this problem several methods have been developed, such as micronization, inclusion in cyclodextrines, the use of inert water-soluble carriers, the use of solid dispersions (WO 00/00179), or nanocrystalline or amorphous forms of active substances.

The effect of the above mentioned methods on the bio-availability often depends on the properties of the active substance. Further the dosage forms developed until now often have drawbacks, such as poor thermodynamic stability, critical or difficult production processes, or poor batch-to-batch reproducibility.

### SUMMARY OF THE INVENTION

It is the object of the present invention to provide an oral formulation for a poorly soluble active substance with a significant increase in bio-availability compared with said active substance in a traditionally formulated form.

A further object of the invention is to provide a formulation which can be prepared using normal formulation procedures and equipment, so that no large capital investment is necessary to produce the formulation.

These and other objects are achieved in accordance with the present invention by providing an oral immediate release formulation with enhanced bio-availability of a poorly water-soluble biologically active substance, wherein said formulation is a homogeneous and thermodynamically stable solid solution comprising as a percentage of the total weight of the formulation:
a) up to 50% by weight of a compound corresponding to formula (I): 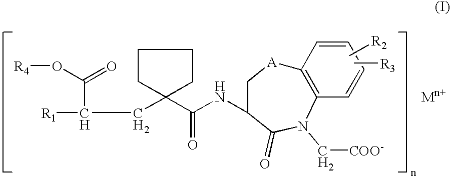
wherein:
   R₁ is selected from the group consisting of (C₁-C₆)alkoxy(C₁-C₆)alkyl which may be substituted by a (C₁-C₆)alkoxy, phenyl-(C₁-C₆)-alkyl and phenyloxy-(C₁-C₆)-alkyl wherein the phenyl group may be substituted with (C₁-C₆)alkyl, (C₁-C₆)alkoxy or halogen, and naphtyl-(C₁-C₆)-alkyl,
   R₂ and R₃ are each independently hydrogen or halogen,
   R₄ is a biolabile ester forming group,
   M is a metal ion, and
   n is 1, 2 or 3;
b) from 20 to 70% by weight of a non-ionic hydrophilic surfactant which is liquid between 15 and 30° C.; and
c) from 5 to 70% by weight of a pharmaceutically acceptable organic polymer or polymer mixture which is liquid above 60° C. and solid below 30° C.
   Optionally, the formulation of the invention further comprises 1 to 10% by weight of a disintegrating agent.

Thus, in accordance with the present invention an oral immediate release formulation with enhanced bio-availability is provided comprising a solid homogeneous and thermodynamically stable solution of a poorly water-soluble biologically active substance, characterised in that the solid solution comprises
a) the active substance in an amount of up to 50% of the total weight of the formulation,
b) a non-ionic hydrophilic surfactant ingredient, which is in the liquid form between 15° and 30° C., in an amount of between 20% and 70% of the total weight of the formulation and
c) a pharmaceutically acceptable organic polymer or mixture of polymers, which polymer or mixture of polymers is in a liquid form above 60° C. and in a solid form below 30° C., in an amount of between 5% and 70% of the total weight of the formulation, and
d) optionally comprises a disintegrating agent in an amount of between 1% and 10% of the total weight of the formulation.

The following definitions are provided to facilitate understanding of certain terms used within the scope of the present application.

Immediate release refers to a release of at least 75% of the drug in a dissolved form from the dosage form within 90 minutes after administration.

Thermodynamically stable refers to the absence of significant physical or chemical changes of the product that might affect the quality of the product during storage for a period up to 5 years under ambient conditions.

Poorly water-soluble means that the aqueous solubility of the active substance is less than 1 in 1000. This means that according to the pharmacopoeial definitions substances that are categorized as “very slightly soluble”, “practically insoluble” and “insoluble” are included in this definition (USP 24/NF 19, page 10; January 2000).

The term non-ionic hydrophilic surfactant refers to those amphiphilic substances that are soluble in water (they have higher HLB values), posses surface activity and are not ionized in aqueous solutions (H. Auterhoff, Worterbuch der Pharmazie, Wissenschafliche Verlagsgesellschaft GmbH, Stuttgart 1981, page 192).

HLB value means a value on a scale from 0 to 20, that is assigned to each surfactant based on the relative proportions of the hydrophilic and hydrophobic part of the molecule. Oil soluble surfactants have low HLB values, whereas water soluble surfactants have higher HLB values. The HLB value is calculated as:
HLB=20(1−Mₒ/M)
in which M is the molecular weight of the molecule and Mₒ is the molecular weight of the hydrophobic part of the molecule.

The ratio between the active substance in the formulation and the non-ionic hydrophilic surfactant is between 1:0.75 and 1:5, preferably between 1:1.5 and 1:4 and most preferably is 1:3. The ratio between the non-ionic hydrophilic surfactant and the pharmaceutically acceptable polymer or mixture of polymers is between 1:4 and 1:0.05, preferably between 1:1.5 and 1:0.1 and most preferably is approximately 1:0.75.

The non-ionic hydrophilic surfactant is preferably selected from the group consisting of polyoxyethylene glycol sorbitan fatty acid esters (polysorbates) and non hydrogentated polyoxyethylene castor oil derivatives, said surfactants having a hydrophilic-lipophilic balance (HLB) value of between 14 and 16.

Polyoxyethylene glycol polysorbates are commercially available from ICI Inc., and are known under the trademark Tween®. For the present invention Tween®) 40, Tween® 60 or Tween® 80 are preferred. The most preferred compound is Tween®) 80. Non hydrogenated polyoxyethylene castor oil derivatives are commercially available from the BASF Corporation under the trademark Cremophor®. The most preferred compound for the present invention is Cremophor® EL.

In one embodiment of the invention, the pharmaceutically acceptable organic polymer or mixture of polymers is comprised primarily of polyethylene glycol (PEG) or a mixture of polyethylene glycols. PEGs are condensation polymers of ethylene oxide, commercially available from Union Carbide Corporation under the trade name Carbowax®. Preferred PEG's are those with a molecular weight of between 1,000 and 10,000 Daltons. More preferred are PEG's having a molecular weight between 4,000 and 6,000 Daltons. The most preferred PEG has a molecular weight of about 6000 Daltons.

In a further embodiment of the invention, the pharmaceutically acceptable organic polymer or mixture of polymers is comprised primarily of polyvinyl pyrrolidone (PVP) or a mixture of polyvinyl pyrrolidones, commercially available from BASF under the trademark Kollidon® having approximate molecular weights of 2,500 up to 3,000,000 Daltons.

In another preferred embodiment of the invention the pharmaceutically acceptable organic polymer or mixture of polymers is comprised primarily of polyvinyl alcohol (PVA) or a mixture of polyvinyl alcohols, commercially available from Shin-Etsu Chemical Co under the trademark Poval® having approximate molecular weights of 30,000 up to 200,000 Daltons.

The formulation optionally comprises a disintegrating agent in an amount of between 1% and 10% of the total weight of the formulation. Normally a disintegration agent is not necessary, but in some cases it may be advantageous to add a small amount of such an agent in order to increase the dissolution of the formulation because of swelling and to increase the water transport into the formulation when contacting the dissolution media. An example of a suitable disintegrating agent is Primojel®, which is commercially available from Penwest Pharmaceuticals. Other disintegrating agents that can be used include Ac-di-Sol®, which is commercially available from FMC, Kollidon CL®, which is commercially available from BASF or Polyplasdone XL®, which is commercially available from ISP.

An especially preferred dosage form for the above formulation is a hard gelatin capsule into which the homogeneous melt mixture is filled and allowed to solidify in situ.

Another dosage form composition is made by filling the melt mixture into soft, elastic gelatin capsules or by forming molded tablets, e.g. by filling the melt mixture into tablet molds, or shaping partially solidified melt mixtures into tablet shapes, for example, by a melt extrusion process like that of Knoll AG, Ludwigshafen, Germany.

The active substances that can be formulated according to the present invention make up a virtually limitless list. As already stated above, the active substances to be formulated are poorly soluble in water, and the invention provides an enhancement of the dissolution properties of the active substances, so that they become more soluble in the substantially aqueous system of the human digestive tract. The active substance is normally used in an amount between about 0.1 and 50% by weight, preferably in an amount between 1 and 50% by weight and more preferably in an amount between about 10 and 50% by weight.

One class of active substances which are poorly soluble in water and for which the present invention is especially useful are the substances described in European patent application no. EP 733,642 which correspond to the formula 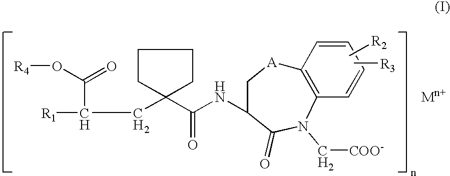
wherein:
   R₁ is a selected from the group consisting of (C₁-C₆)alkoxy(C₁-C₆)alkyl which may be substituted by a (C₁-C₆)alkoxy, phenyl-(C₁-C₆)-alkyl and phenyloxy-(C₁-C₆)-alkyl wherein the phenyl group may be substituted with (C₁-C₆)alkyl, (C₁-C₆)alkoxy or halogen, and naphtyl-(C₁-C₆)-alkyl,
   R₂ and R₃ are both independently hydrogen or halogen,
   R₄ is a biolabile ester forming group,
   M is a metal ion, preferably a bivalent metal ion,
   n is 1, 2 or 3.

(C₁-C₄)-alkyl is defined as a straight or branched alkyl group consisting of between 1 and 4 carbon atoms. (C₁-C₄)-alkoxy is defined as a straight or branched alkoxy group consisting of between 1 and 4 carbon atoms.

The present invention also relates to a solid solution formulation as described above of a poorly water soluble compound of formula 1. M is preferably a Li⁺, Mg²⁺, Zn²⁺ or a Ca²⁺ ion and most preferrably a Ca²⁺ ion. R₁ is preferably phenylethyl. R₂ and R₃ are preferably hydrogen, and R₄ is preferably ethyl. The preferred compound is the calcium salt of 1H-1-Benzazepine-1-acetic acid, 3-[[[1-[2-(ethoxycarbonyl)-4-phenylbutyl]cyclopentyl]carbonyl]amino]-2,3,4,5-tetrahydro-2-oxo-. The most preferred compound is said compound in its 3S,2′R form. This compound is referred to as Compound S-Ca. The corresponding acid (1H-1-Benzazepine-1-acetic acid, 3-[[[1-[2-(ethoxycarbonyl)-4-phenylbutyl]cyclopentyl]carbonyl]amino]-2,3,4,5-tetrahydro-2-oxo-) is referred to as Compound S-H, and the corresponding S-α-methylbenzylamine salt is referred to as Compound S-Mba.

The formulation described above can be prepared using conventional formulation procedures and equipment. Therefore it is another aspect of the present invention to provide a method of preparing a formulation as described above, in which a) the non-ionic hydrophilic surfactant is mixed with the pharmaceutically acceptable organic polymer or mixture of polymers at between 50-100° C., preferably between 60 and 70° C., b) the active ingredient is added and dissolved at said temperature, c) the resulting mixture is optionally filled into a capsule, and d) the resulting mixture is solidified at room temperature.

Alternatively the non-ionic hydrophilic surfactant, the pharmaceutically acceptable organic polymer or mixture of polymers and the active substances are mixed together and heated to a temperature of between 50 and 100° C., preferably between 60 and 70° C., until a clear solution is obtained, optionally followed by filling the solution into a capsule.

### BRIEF DESCRIPTION OF THE DRAWINGS

FIG. 1 is a graph showing the effect of the amount of surfactant on the rate of poorly-soluble active ingredient release from a composition according to the invention;

FIG. 2 is another graph showing the effect of the amount of surfactant on the rate of active ingredient release;

FIG. 3 is a graph showing the effect of different polyethylene glycols on the rate of release of poorly-soluble active ingredient from compositions according to the invention;

FIG. 4 is another graph showing the effect of different polyethylene glycols on the rate of active ingredient release;

FIG. 5 is a graph showing the effect of different cations on the rate of poorly-soluble active ingredient releast from compositions according to the invention; and

FIG. 6 is a graph showing the results of a bioavailability cross-over study with formulations according to the invention.

### EXAMPLES

The following examples are intended to illustrate the invention in further detail without restricting the scope of the invention in any way.

### Example 1

### Effect of Type and Quantity of Surfactant on Release

Preparation of the Formulations.

The non-ionic hydrophilic surfactant, Tween 80 or Cremophor EL was heated together with a hydrohilic polymer, PEG 6000 up to a temperature above 60° C. The active substance was added and dissolved in the melt at said temperature. The resulting solution was filled into size 0 (zero) capsules. The solution solidified in the capsule at room temperature.

Increasing quantities of a surfactant together with a poorly water soluble active substance and the hydropholic polymer were composed. The compositions are given in Table 1 for liquid filled capsules which contain 50 mg of the active substance. The effect of the amount and the effect of type of surfactant on the release of the active substance from the liquid filled capsule was determined.

TABLE 1
Effect of quantity and type of surfactant on the dissolution of the
active substance (Amounts are expressed in %)

| | Composition in % | | | | | | |
|---|---|---|---|---|---|---|---|
| Materials | A | B | C | D | E | F | G |
| Active substance^{*)} | 16.0 | 16.0 | 16.0 | 16.0 | 16.0 | 16.0 | 16.0 |
| Tween 80 ® | 0.0 | 12.0 | 24.0 | 48.0 | n.a. | n.a. | n.a. |
| Cremophor EL ® | 0.0 | n.a. | n.a. | n.a. | 12.0 | 24.0 | 48.0 |
| PEG 6000 | 84.0 | 72.0 | 60.0 | 36.0 | 72.0 | 60.0 | 36.0 |

*calcium salt of 1H-1-Benzazepine-1-acetic acid, 3-[[[1-[(2R)-2-(ethoxycarbonyl)-4-phenylbutyl]cyclopentyl]carbonyl]amino]-2,3,4,5-tetrahydro-2-oxo-, (3S)-. (Compound S—Ca)
n.a.: not applicable

In-Vitro Dissolution Testing
Dissolution System.

Dissolution testing of the liquid filled capsules was performed in artificial gastrointestinal fluids at 37° C. using USP II apparatus at a paddle speed of 100 rpm and a sinker for each capsule The dissolution was tested over a sequential range of increasing pH of the medium starting with 400 ml at pH 2, prepared from 400 ml 0.01 N hydrochloric acid. One hour after starting the dissolution 15 ml of the medium was withdrawn, and the pH of the buffer was changed to pH 4.5 by adding 88.5 ml 0.05 N glacial acetic acid and 211.5 ml 0.05 N sodium acetate solution. After 30 minutes 5 ml of the medium was withdrawn, and the pH of the buffer was adjusted to pH 6.8 by adding 180 ml 0.2 N disodium hydrogen phosphate and 120 ml 0.2 N potassium dihydrogen phosphate. After 21/2 hours, 5 ml of the medium was withdrawn, and the dissolution test was stopped.

Chromatographic System.

A high-performance liquid chromatographic system was used equipped with a thermostated column compartment, a UV-absorbance detector with adjustable wavelength and an integrating system. The analytical column (length 3 cm, internal diameter 3 mm) was a C18-modified silica, preferably Inertsil® ODS-3 column, particle size 3 μm. The mobile phase was a degassed mixture of 350 ml of water containing 800 mg of ammonium acetate and 800 μl of trifluoracetic acid and 650 ml of acetonitrile. The flow rate was 0.5 ml/min. The column temperature was 40° C. The injection volume was 5 μl, and the wavelength of the UV absorbance detector was 236 nm. For external standardization 0.12 mg of Compound S-Mba RS was dissolved in 1 ml of the mobile phase.

The quantity of dissolved Compound S-H, expressed in percent relative to the label claim, is given by the equation 1:

$$\% dissolved = \frac{{I}_{sa} \times {m}_{st} \times {V}_{sa} \times C \times 0.8152}{{I}_{st} \times {V}_{st} \times L C}$$

Equation 1: Calculation of the quantity dissolved Compound S-H.
in which:
Iₛₜ=peak area of Compound S-H in the standard chromatogram;
Iₛₐ=peak area of Compound S-H in the sample chromatogram;
Vₛₜ=dilution volume of the standard solution, in ml (=50 ml);
Vₛₐ=dilution volume of the sample solution, in ml (=400, 700 and 1000 ml);
mₛₜ=weighed quantity of Compound S-Mba RS, in mg;
C=purity of Compound S-Mba RS, in % m/m;
LC=label claim of the analysed capsule, expressed as Compound S-H.
0.8152=ratio between the molecular masses of the Compound S-H and Compound S-Mba.

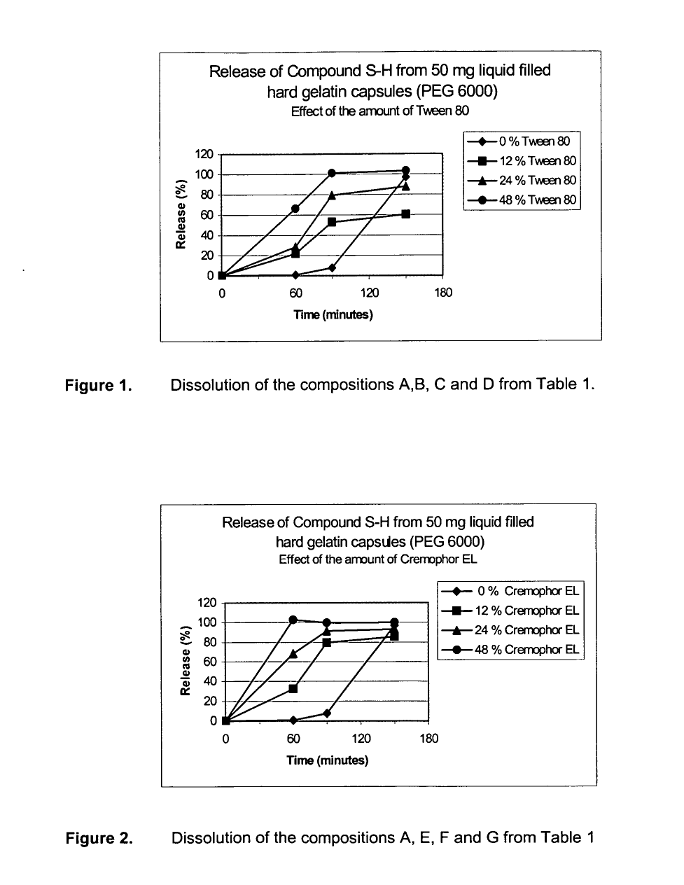

The dissolution results of the formulations A, B, C and D (see Table 1), containing Tween 80 as surfactant as determined by the above mentioned HPLC method are given in FIG. 1 (♦=0%, ▪=12%; ▴=24% and ●=48% Tween 80). The dissolution results of the formulations E, F and G (see Table 1) containing Cremophor EL as surfactant are given in FIG. 2 (♦=0%; ▪=12%; ▴=24% and ●=48% Chremophor EL).

From the results given in FIGS. 1 and 2, it can be seen that the release of the active substance from liquid filled capsules is determined by the amount of hydrophilic surfactant used in the composition. The amount of released active substance increases with increasing amounts of surfactant.

More specifically it can be seen that the release of the active substance at pH 2 (release data during the first 60 minutes of the dissolution test) is determined by the amount of surfactant in the composition. The pH change from 2 to 4.5 (release data during the next 30 minutes) improves the release of the active substance from the compositions which contain the hydrophilic surfactant at a low level. At the end of the pH 5 period it is observed that the active substance is completely dissolved when the composition contains at least 12% surfactant Cremophor EL or 24% Tween 80. Finally the pH change from 4.5 to 6.8 does not influence the release data anymore. The active substance remains completely dissolved when using at least 12% Cremophor EL or 24% Tween 80.

### Example 2

### Effect of Type of Hydrophilic Polymer on Release

The hydrophilic polymer in the liquid filled capsules can be a polyethylene glycol product. The influence of the molecular weight of this polymer on the dissolution was tested in the compositions shown in Table 2. The formulations were prepared as described in Example 1.

TABLE 2
Composition with different types of polyethylene
glycol (n.a. = not applicable)

| | Composition (%) | | |
|---|---|---|---|
| Material | D | H | J |
| Active substance* | 16 | 16 | 16 |
| Tween 80 | 48 | 48 | 48 |
| PEG 4000 | n.a. | 36 | n.a. |
| PEG 6000 | 36 | n.a. | n.a. |
| PEG 50000 | n.a. | n.a. | 36 |

*Compound S—Ca;
n.a. = not applicable

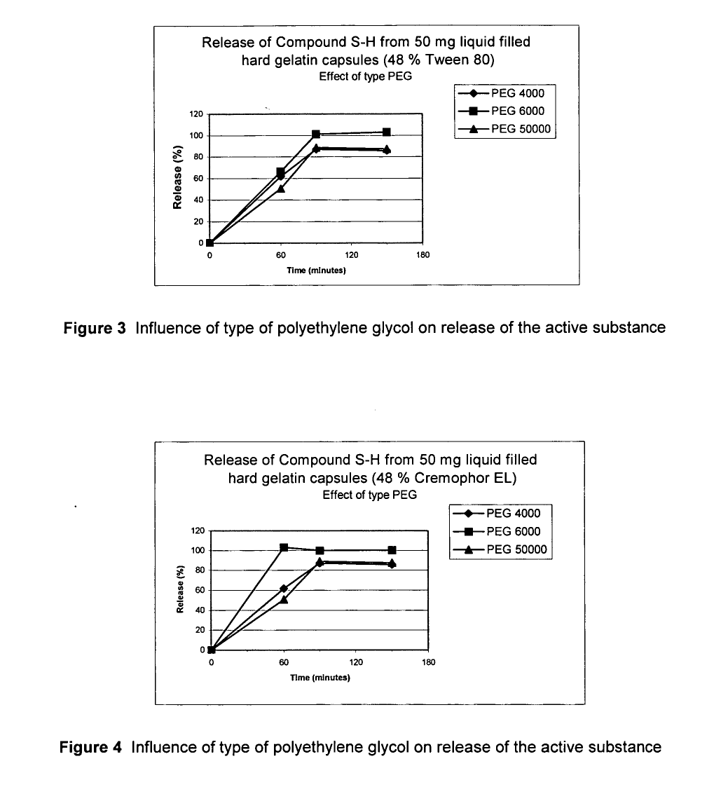

Dissolution testing was carried out as described in Example 1. The dissolution results of the composition with Tween 80 and with different types of polyethylene glycol are given in FIG. 3 (♦=PEG 4000; ▪=PEG 6000; ▴=PEG 50000). It can be seen that the active substance release from the composition containing PEG 4000 and PEG 50000 is comparable, but delayed in comparison with PEG 6000, without, however, being sustained release formulations.

The most preferred hydrophilic polymer is PEG 6000 because PEG 4000 will cause leakage from the capsules sooner due to its lower melting point. On the other hand PEG 50000 is difficult to handle because of the relatively high viscosity of this material in the molten phase.

The influence of the type of hydrophilic polymer was also demonstrated with capsule formulations containing Cremophor EL as surfactant. The surfactant Tween 80 of the previous examples, composition D, H and J was replaced with the same amount of Cremophor EL. The dissolution test was carried out as described previously. The dissolution results of liquid filled capsules containing 48% Cremophor EL are given in FIG. 4 (♦=PEG 4000; ▪=PEG 6000; ▴=PEG 50000). FIG. 4 clearly shows that the active substance release from liquid filled capsules containing the hydrophilic polymers PEG 4000 and PEG 50000 is comparable but delayed in comparison with PEG 6000. The formulations cannot, however, be regarded as sustained release formulations.

### Example 3

### Effect of Different Cations on the Release of the Active Substance

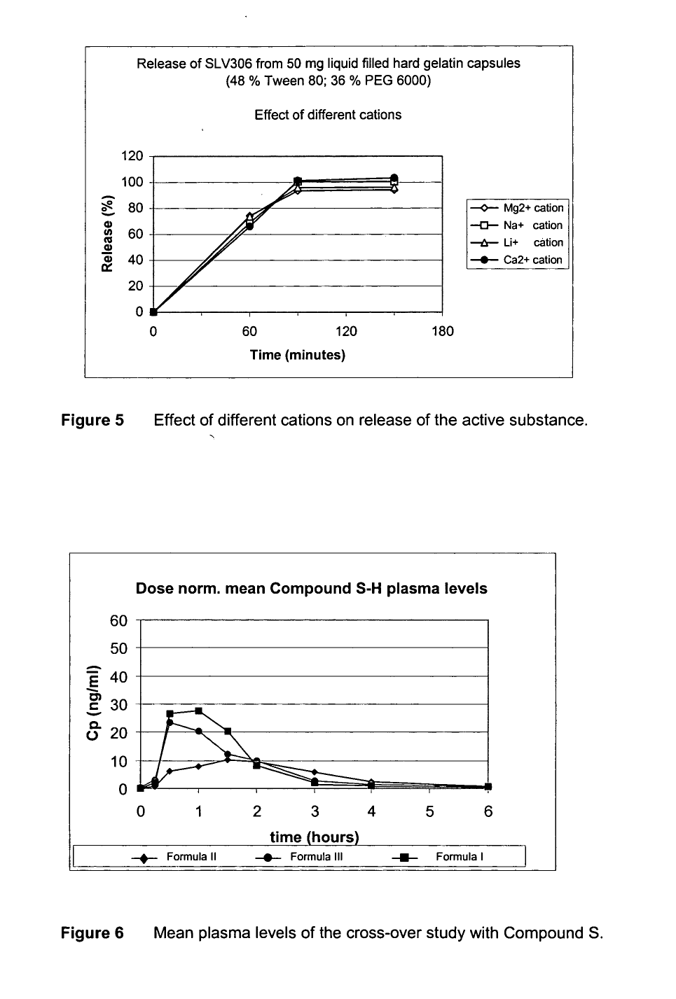

The most preferred Ca²⁺ ion in the formula of the active substance was replaced by several other metal ions like Mg²⁺, Na⁺ and Li⁺. These active substances were formulated according to composition D in Table 1. This means the formulations contain 16% active substance, 48% Tween 80 and 36% PEG 6000. The formulations were prepared in accordance with the method described for Example 1. The dissolution results of the active substance with the Ca²⁺, Mg²⁺, Na⁺ or Li⁺ ion are given FIG. 5. (●=Ca²⁺, ⋄=Mg²⁺, □=Na⁺ or Δ=Li⁺). From the dissolution results it can be seen that these cations do not affect the release of the active substance. The profiles at pH 2, pH 4.5 and pH 6.8 are comparable.

### Example 4

### Bioavailability Study

A cross-over study in 15 male subjects was performed to test the bioavailability of the liquid filled hard gelatin capsule. Compound S-Ca (Formulation I and III) or Compound S-H (Formulation II) was used as the drug substance.

The subjects were administered the following formulations: (I) 2×103.7 mg compound S-Ca (corresponding to 100 mg compound S-H) liquid filled in hard gelatin capsule prepared according to Example 1 with the composition D, (II) 2×100 mg compound S-H in hard gelatin capsule as a 25% m/m powder blend on tricalcium phosphate, (III) 8×25 mg plain tablet, consisting of 25.94 mg Compound S-Ca (corresponding to 25 mg compound S-H), 172 mg of microcrystalline cellulose PH101, 172 mg of mannitol-25, 8 mg of hydroxypropyl methyl cellulose E5, 20 mg of sodium starch glycolate and 2 mg of sodium stearyl fumarate. From the mean plasma levels shown in FIG. 6 the results as given in Table 3 are obtained.

TABLE 3
Results of cross-over study in 15 male subjects

| Formulation | Cmax ratio | Relative bioavailability |
|---|---|---|
| I | 2.7 | 1.8 |
| II | 1 | 1 |
| III | 1.9 | 1.5 |

The results from Table 3 indicate that the bio-availability of the drug substance from the liquid filled hard gelatin capsule containing 103.7 mg of Compound S Ca, 311 mg Tween 80 and 234 mg polyethylene glycol 6000 improves with 80% in comparison with the bio-availability of formulation III in which the Compound S-H is adsorbed on tricalcium phosphate as a 25% m/m powder blend and with 20% in comparison with the plain tablet of Compound S-Ca.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. An oral immediate release formulation of a poorly water-soluble biologically active substance with enhanced bio-availability, wherein said formulation is a homogeneous and thermodynamically stable solid solution comprising as a percentage of the total weight of the formulation:
a) up to 50% by weight of a compound corresponding to formula (I): 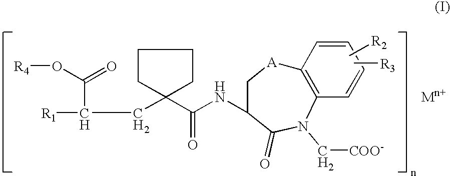
wherein:
R₁ is selected from the group consisting of (C₁-C₆)alkoxy(C₁-C₆)alkyl which may be substituted by a (C₁-C₆)alkoxy, phenyl-(C₁-C₆)-alkyl and phenyloxy-(C₁-C₆)-alkyl wherein the phenyl group may be substituted with (C₁-C₆)alkyl, (C₁-C₆)alkoxy or halogen, and naphtyl-(C₁-C₆)-alkyl,
R₂ and R₃ are each independently hydrogen or halogen,
R₄ is a biolabile ester forming group,
M is a metal ion, and
n is 1, 2 or 3;
b) from 20 to 70% by weight of a non-ionic hydrophilic surfactant which is liquid between 15 and 30° C.; and
c) from 5 to 70% by weight of a pharmaceutically acceptable organic polymer or polymer mixture which is liquid above 60° C. and solid below 30° C.

2. A formulation according to claim 1, further comprising 1 to 10% by weight of a disintegrating agent.

3. A formulation according to claim 1, wherein M is a bivalent metal ion.

4. A formulation according to claim 1, wherein the active substance and the non-ionic hydrophilic surfactant are present in a ratio of between 1:0.75 and 1:5, and the surfactant and the pharmaceutically acceptable organic polymer or polymer mixture are present in a ratio of between 1:4 and 1:0.05.

5. A formulation according to claim 4, wherein the ratio between the surfactant and the pharmaceutically acceptable organic polymer or polymer mixture is between 1:1.5 and 1:0.1.

6. A formulation according to claim 1, wherein the non-ionic hydrophilic surfactant is selected from the group consisting of polyoxyethylene glycol sorbitan fatty acid esters and non hydrogenated polyoxyethylene castor oil derivatives, said non-ionic hydrophilic surfactant having a hydrophilic-lipophilic balance (HLB) value of between 14 and 16.

7. A formulation according to claim 6, wherein the non-ionic hydrophilic surfactant ingredient is a polyoxyethylene glycol sorbitan fatty acid ester.

8. A formulation according to claim 1, wherein the pharmaceutically acceptable organic polymer is a polyethylene glycol or a mixture of polyethylene glycols, each with a molecular weight of between 1000 and 50000 Daltons.

9. A formulation according to claim 8, wherein the polyethylene glycol or polyethylene glycols each have a molecular weight of between 4000 and 10000 Daltons.

10. A formulation according to claim 1, wherein the pharmaceutically acceptable organic polymer or polymer mixture is a polyvinyl pyrrolidone or a mixture of polyvinyl pyrrolidones with molecular weight range of 2,500 up to 3,000,000 Daltons or a polyvinyl alcohol or a mixture of polyvinyl alcohols with molecular weight range of 30,000 up to 200,000 Daltons.

11. A formulation according to claim 1, wherein M is Ca²⁺.

12. A formulation according to claim 1, wherein said poorly water-soluble active substance is the calcium salt of 3-[[[1-[2-(ethoxycarbonyl)-4-phenylbutyl]-cyclopentyl]carbonyl]-amino]-2,3,4,5-tetrahydro-2-oxo-1H-1-benzazepine-1-acetic acid.

13. A formulation according to claim 12, wherein the active substance is in the 3S,2′R form.

14. A formulation according to claim 1, contained in a hard gelatine or soft gelatine capsule.

15. A method of preparing a formulation according to claim 1, comprising:
a) mixing the non-ionic hydrophilic surfactant with the pharmaceutically acceptable organic polymer or polymer mixture at between 50-100° C.;
b) adding the compound of formula (I) to the mixture from a) and dissolving the compound of formula (I) in the mixture from a) at said temperature; and
c) solidifying the mixture from b) at room temperature.

16. A method according to claim 15, wherein the mixture from b) is filled into a capsule before it is solidified.

17. A method of preparing a formulation according to claim 1, comprising:
a) mixing the non-ionic hydrophilic surfactant with the pharmaceutically acceptable organic polymer or polymer mixture and the compound of formula (I) at between 50-100° C., and
b) solidifying the mixture from a) at room temperature.

18. A method according to claim 17, wherein the mixture from a) is filled into a capsule before it is solidified.
